SEQUENCE LISTING (2 sequences)

SEQ ID NO: 1 - protein, 23 aa, Styela clava
FLRFIGSVIHGIGHLVHHIGVAL

SEQ ID NO: 2 - protein, 23 aa, Artificial Sequence
  artificial peptide
FLRFIGSVKHGKGHLVHHIGVAL

(19) United States
(12) Patent Application Publication
(10) Pub. No.: US 2019/0038714 A1
(43) Pub. Date: Feb. 7, 2019

(54) ANTI-INFLAMMATORY COMPOSITION COMPRISING CLAVASPIRIN PEPTIDE ANALOGUE AS EFFECTIVE INGREDIENT

(71) Applicant: INDUSTRY-ACADEMIC COOPERATION FOUNDATION, CHOSUN UNIVERSITY, Gwangju (KR)

(72) Inventors: Yoonkyung PARK, Gwangju (KR); Jong-Kook LEE, Jeollanam-do (KR)

(21) Appl. No.: 15/757,689

(22) PCT Filed: Sep. 12, 2016
(86) PCT No.: PCT/KR2016/010292

§ 371 Date: Mar. 6, 2018

(30) Foreign Application Priority Data
Sep. 15, 2015 (KR) 10-2015-0130341

(51) Int. Cl. A61K 38/17; A61P 31/04; A61P 29/00; A23L 33/18

(52) U.S. Cl. CPC A61K 38/1767; A61P 31/04; A23V 2002/00; A23L 33/18; A61P 29/00

(57) ABSTRACT

An anti-inflammatory composition includes a clavaspirin peptide analogue as an effective ingredient. CSP-4 peptide as a synthetic peptide produced by substituting the 9th and 12th amino acids of clavaspirin peptide originating from stalked sea squirt (Styela clava) with positively charged lysine (K) has an excellent anti-inflammatory effect against an animal model with inflammation response that is caused by antibiotics-resistant bacteria, and exhibits almost no cytotoxicity. Thus, CSP-4 peptide as a clavaspirin peptide analogue of the present invention can be advantageously used as an effective ingredient of a pharmaceutical composition, a cosmetic composition, a food additive, or the like for preventing or treating an inflammatory disorder.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2016/010292, filed Sep. 12, 2016, which claims priority to the benefit of Korean Patent Application No. 10-2015-0130341 filed in the Korean Intellectual Property Office on Sep. 15, 2015, the entire contents of which are incorporated herein by reference.

### TECHNICAL FIELD

The present invention relates to an anti-inflammatory composition comprising a clavaspirin peptide analogue as an effective ingredient.

### BACKGROUND ART

Decreased physical activity resulting from economic growth yields poor immunity, and human beings are now exposed to a risk of various diseases as caused by infection of microbes like bacteria, fungi, or parasites. Although various kinds of pharmaceuticals are currently used all over the world to prevent and treat diseases, pathogen resistance is rather enhanced by misuse and abuse of pharmaceuticals. In particular, an increase of VRE (vancomycin-resistant enterococci), MRSA (methicillin-resistant Staphylococcus aureus), and MDRP (multidrug-resistant pathogens), which are referred to as superbugs, is a subject of global attention.

Bacterial infection is one of the most common and deadly causes of a human disease. However, due to abuse of antibiotics, bacterial resistance to antibiotics has been yielded unfortunately. The rate of exhibiting resistance to antibiotics by bacteria is indeed much faster than the rate of developing new antibiotics analogues. For example, various bacterial species like MRSA, Enterococcus faecalis, Mycobacterium tuberculosis, and Pseudomonas aeruginosa, which may pose a threat to human life, have developed resistance to all antibiotics that are known until now.

Tolerance to the antibiotics is a phenomenon which is distinguished from the resistance to the antibiotics. Ever since found from MRSA, it provides an important key for studying the working mechanism of penicillin. Bacterial species exhibiting the tolerance show growth stall in the presence of antibiotics at common concentration, but without any death eventually. The tolerance is caused due to a lack of the activity of an autolytic bacterial enzyme like autolysin as the antibiotics inhibit an enzyme for synthesizing cell wall, and this leads to the results that, as an endogenous hydrolytic enzyme is activated by penicillin, bacterial cell death is caused, and the bacteria also suppress the enzyme activity to survive even under a treatment with antibiotics.

Having tolerance to various antibiotics by bacteria is clinically very important, because, once it becomes impossible to eradicate bacteria with tolerance, usefulness of a clinical treatment with antibiotics for infection is impaired. Furthermore, having tolerance is believed to a prerequisite requirement for developing resistance to antibiotics, and that is because there are bacterial strains which manage to survive even after a treatment with antibiotics. By acquiring new genetic elements to exhibit resistance to antibiotics, those bacterial strains keep growing even in the presence of the antibiotics. Since all bacteria exhibiting resistance are indeed known to have tolerance too, it is necessary to develop novel antibiotics which can be used for eradicating those bacteria having resistance to antibiotics.

In terms of a working mechanism, the tolerance broadly consists of two pathways. The first pathway is phenotypic tolerance which occurs during every bacteria growth with decreasing rate, and the second pathway is genetic tolerance caused by mutation which occurs in specific types of bacteria. In all of those cases, the basic phenomenon is an occurrence of down regulation of autolysin activity. This down regulation is transient in case of phenotypic tolerance against external stimulation, while it is permanent in case of genetic tolerance in which there is an occurrence of a mutation for causing a change in pathway for regulating cell lysis. The simplest genetic tolerance is based on a defect in autolysin enzyme, and due to various kinds of reasons which have not been clarified, a bacterial strain having the tolerance as caused by a defect in autolysin has not been clinically found yet, and clinical tolerance is rather achieved by regulating the activity of autolysin.

As discussed in the above, in order to deal with bacteria which exhibit resistance to antibiotics, development of new antibiotics is required, and also development of new antibiotics which work independently of the activity of autolysin is required.

Meanwhile, when tissues (cells) are damaged or infected with external infectious sources (e.g., bacteria, fungi, viruses, and various kinds of allergy triggering substances), the inflammatory response yields, as being involved with various inflammation-mediating factors and immune cells present in peripheral blood vessels and body fluid, a series of complex physiological responses such as enzyme activation, secretion of inflammation-mediating substances, body fluid invasion, cell migration, or tissue damage and also external symptoms such as erythema, swelling, heat, or pain. In normal cases, external infectious sources are removed, and damaged tissues are regenerated by an inflammatory response to exhibit the function of recovering the normal activity of a living organism. However, if the inflammatory response is either excessive or continues as the antigens are not removed or there are some internal substances to cause an inflammatory response, it instead becomes a main pathological phenomenon of a disorder (e.g., hypersensitive disease, chronic inflammation, or the like) and may serve as an obstacle during a treatment process like blood transfusion, drug administration, and organ transplantation.

Until now, a treatment is carried out by administering a pharmaceutical preparation for reducing physical inconveniences of an inflammatory response. However, since common anti-inflammatory pharmaceuticals are used for a treatment of a broad range of disorders and different disorders are often treated with the same pharmaceutical, the treatment effect and side effect are shared among them. Due to the side effect, however, such pharmaceutical cannot be used for a long period of time. As a result, the therapy employed until now has a significant potential of side effect. Thus, development of a new or improved therapeutic agent is both essential and urgent, and there is a demand for an anti-inflammatory agent which is free of any side effect problem and can be used safely for a human body.

In Korean Patent Registration No. 1158368, “Composition for anti-inflammatory activity containing sweetfish proteins and peptides and extraction method therefor” is disclosed, and in Korean Patent Application Publication No. 2015-0085935, “CMA3 analogue peptide derived from CM-MA peptide and uses thereof” is disclosed. However, the anti-inflammatory composition containing a clavaspirin peptide analogue as an effective ingredient as described in the present invention has never been disclosed before.

### SUMMARY

The present invention is devised under the circumstances described above. Specifically, inventors of the present invention found that the synthetic peptide produced by substituting the 9^{th} and 12^{th} amino acids of clavaspirin peptide, which consists of 23 amino acids, with lysine (K) has an excellent anti-inflammatory effect against an animal model with inflammation response that is caused by antibiotics-resistant bacteria, and exhibits almost no cytotoxicity. The present invention is completed accordingly.

In order to solve the problems described above, the present invention provides a functional health food composition for preventing or alleviating an inflammatory disorder which comprises, as an effective ingredient, a clavaspirin peptide analogue consisting of the amino acid sequence of SEQ ID NO: 2.

The present invention further provides a pharmaceutical composition for preventing or treating an inflammatory disorder which comprises, as an effective ingredient, a clavaspirin peptide analogue consisting of the amino acid sequence of SEQ ID NO: 2.

The present invention further provides an anti-inflammatory composition which comprises, as an effective ingredient, the aforementioned clavaspirin peptide analogue.

The present invention further provides a cosmetic composition for preventing or alleviating an inflammatory disorder which comprises, as an effective ingredient, the aforementioned clavaspirin peptide analogue.

The present invention still further provides an antimicrobial composition for use against one or more selected from a group consisting of Staphylococcus aureus, Bacillus subtilis, and MRSA (methicillin-resistant Staphylococcus aureus) which comprises, as an effective ingredient, the aforementioned clavaspirin peptide analogue.

The clavaspirin peptide analogue of the present has an excellent anti-inflammatory effect against an animal model with inflammation response that is caused by antibiotics-resistant bacteria, and exhibits almost no cytotoxicity. As such, it can be advantageously used as an effective ingredient of a pharmaceutical composition, a cosmetic composition, a food additive, or the like for preventing or treating an inflammatory disorder.

### BRIEF DESCRIPTION OF THE DRAWINGS

FIG. 1 shows the result of determining erythrocyte lysis activity of CSP-4 peptide of the present invention in which data are expressed for control group (melittin, ◯) and CSP-4 (●).

FIGS. 2A and 2B show the results of determining the expression of TNF-α and IL-1β in Raw 264.7 as phagocytes. The results are shown in terms of mean value±standard deviation in which the mean value is an average of 3 individual tests.

FIGS. 3A to 3E show the results of determining the expression after treating the skin of a hairless mouse, which has dermatitis caused by S. aureus CCARM 0027, with CSP-4 peptide, in which the determination is made 7 days after the treatment, or, after collecting the tissue (FIG. 3A), by H&E staining (FIG. 3B) or immunohistochemical method following binding of TLR-2 (FIG. 3C), TNF-α (FIG. 3D), or IL-1β (FIG. 3E). In the FIG. 3A, 1) represents a control group, 2) represents CSP-4 200 μg/ml, 3) represents S. aureus CCARM 0027, 4) represents S. aureus CCARM 0027+CSP-4 100 μg/ml, and 5) represents S. aureus CCARM 0027+CSP-4 200 μg/ml.

FIG. 4 shows the result of determining a change in the skin of a hairless mouse under an electron scanning microscope (SEM). In FIG. 4, A) represents a control group, B) represents CSP-4 200 μg/ml, C) represents S. aureus CCARM 0027, D) represents S. aureus CCARM 0027+CSP-4 100 μg/ml, and E) represents S. aureus CCARM 0027+CSP-4 200 μg/ml.

FIG. 5 shows the result of determining the expression of cytokines and inflammation-exhibiting proteins in the skin of a hairless mouse with induced inflammation, in which the expression is determined also in the presence of the peptide of the present invention. In the figure, A) represents a control group, B) represents CSP-4 200 μg/ml, C) represents S. aureus CCARM 0027, D) represents S. aureus CCARM 0027+CSP-4 100 μg/ml, and E) represents S. aureus CCARM 0027+CSP-4 200 μg/m

### DETAILED DESCRIPTION

To achieve the purpose of the present invention, the present invention provides a functional health food composition for preventing or alleviating an inflammatory disorder which comprises, as an effective ingredient, a clavaspirin peptide analogue consisting of the amino acid sequence of SEQ ID NO: 2.

Clavaspirin as a mother peptide known to have the amino acid sequence of SEQ ID NO: 1 is an antimicrobial peptide originating from stalked sea squirt (Styela clava), and it can be produced by a common peptide synthesis method that is known in the pertinent art. The production method is not particularly limited.

The clavaspirin peptide analogue as an effective ingredient of the present invention is also referred to as CSP-4 and has the amino acid sequence of SEQ ID NO: 2.

According to one embodiment of the present invention, provided is a CSP-4 peptide having the amino acid sequence of SEQ ID NO: 2 that is prepared by substituting two isoleucines (I) as the 9^{th} and the 12^{th} amino acids of the amino acid sequence of SEQ ID NO: 1 as a mother peptide with lysine (K) having less hydrophobicity based on a solution phase peptide synthesis by Merrifield.

CSP-4 peptide having the amino acid sequence of SEQ ID NO: 2 of the present invention can be produced by a common peptide synthesis method that is known in the pertinent art, and the production method is not particularly limited. The 9^{th} and the 12^{th} amino acids that are substituted in the present invention can be also substituted with, other than lysine (K), arginine (R) or histidine (H) which are a positively charged amino acid, and they are preferably lysine (K).

The term “inflammation” encompasses any inflammatory response occurring in a human body with no limitation, and it also encompasses acute and/or chronic inflammatory disorder as well as a disorder accompanying inflammation.

The inflammation may be induced by bacteria, and the bacteria are preferably Gram-negative bacteria, Gram-positive bacteria, or antibiotics-resistant bacteria. More preferably, Gram-negative bacteria are Escherichia coli or Proteus vulgaris, Gram-positive bacteria are Listeria monocytogenes, Staphylococcus epidermidis, Bacillus subtilis, or Staphylococcus aureus, and the antibiotics-resistant bacteria are MRSA (methicillin-resistant Staphylococcus aureus), but they are not limited thereto.

According to one embodiment of the present invention, the inflammation inhibiting effect was confirmed by treating a mouse, which has been induced to have inflammation by S. aureus MRSA 0027 as bacteria with antibiotics resistance, with CSP-4 peptide of the present invention.

According to one embodiment of the present invention, the composition inhibits the expression of cytokines or inflammation-related proteins, and preferably the expression of TNF-α, IL-1β, NF-κB or TLR-2 (Toll-like receptor 2), but not limited thereto.

The food is not particularly limited as long as it is food which can be taken to reduce the inflammation.

With regard to the functional health food composition according to one embodiment of the present invention, the inflammatory disorder may be at least one selected from a group consisting of dermatitis, allergy, atopy, asthma, conjunctivitis, periodontitis, rhinitis, otitis media, pharyngitis, tonsillitis, pneumonia, stomach ulcer, gastritis, Crohn's disease, colitis, gout, ankylosing spondylitis, rheumatic fever, lupus, fibromyalgia, psoriatic arthritis, osteoarthritis, rheumatic arthritis, shoulder periarthritis, tendinitis, tenosynovitis, tendonitis, myositis, hepatitis, cystitis, nephritis, Sjogren's syndrome, multiple sclerosis, and acute or chronic inflammatory disorder, but it is not limited thereto.

When CSP-4 peptide of the present invention is used as a food additive, CSP-4 peptide may be directly added or used with other food or food components, and it can be suitably used according to a general method. Blending amount of an effective ingredient can be suitably determined depending on the purpose of use (e.g., prophylaxis, health management, or therapeutic treatment). In general, for producing a food product or a beverage, CSP-4 peptide of the present invention is added in an amount of 15 parts by weight or less, and preferably 10 parts by weight or less relative to peptide raw materials. However, in case of an application for a long period of time like achieving health and hygiene or health management, the blending amount may be lower than the aforementioned range. As there is no problem in terms of the stability, the effective ingredient may be used in an amount that is higher than the aforementioned range.

Type of the food is not particularly limited. Examples of the food to which the peptide can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all health foods in general sense are included therein.

The health beverage composition of the present invention may contain, like common beverages, various flavors or natural carbohydrates as an additional component. Examples of the natural carbohydrates include monosaccharides such as glucose or fructose, disaccharides such as maltose or sucrose, polysaccharides such as dextrin or cyclodextrin, and sugar alcohols such as xylitol, sorbitol, or erythritol. As a sweetening agent, a natural sweetening agent such as thaumatin or stevia extract or a synthetic sweetening agent such as saccharine and aspartame can be used.

Other than those described above, CSP-4 peptide of the present invention may contain various kinds of a nutritional agent, vitamins, electrolyte, flavors, a coloring agent, pectinic acid and salts thereof, alginic acid and salts thereof, organic acids, a protective colloid thickening agent, a pH adjusting agent, a stabilizing agent, a preservative, glycerin, alcohol, and a carbonating agent used for carbonate drink.

The present invention further provides a pharmaceutical composition for preventing or treating an inflammatory disorder which comprises, as an effective ingredient, a clavaspirin peptide analogue consisting of the amino acid sequence of SEQ ID NO: 2.

CSP-4 peptide having the amino acid sequence of SEQ ID NO: 2 in the present invention can be produced by a common peptide synthesis method that is known in the pertinent art. The production method is not particularly limited.

The inflammation may be induced by bacteria, and the bacteria are preferably Gram-negative bacteria, Gram-positive bacteria, or antibiotics-resistant bacteria. More preferably, Gram-negative bacteria are Escherichia coli or Proteus vulgaris, Gram-positive bacteria are Listeria monocytogenes, Staphylococcus epidermidis, Bacillus subtilis, or Staphylococcus aureus, and the antibiotics-resistant bacteria are MRSA (methicillin-resistant Staphylococcus aureus), but they are not limited thereto.

According to one embodiment of the present invention, the inflammation inhibiting effect was confirmed by treating a mouse, which has been induced to have inflammation by S. aureus MRSA 0027 as bacteria with antibiotics resistance, with CSP-4 peptide of the present invention.

According to one embodiment of the present invention, the composition inhibits the expression of cytokines or inflammation-related proteins, and preferably the expression of TNF-α, IL-1β, NF-κB or TLR-2, but not limited thereto.

With regard to the pharmaceutical composition according to one embodiment of the present invention, the inflammatory disorder may be at least one selected from a group consisting of dermatitis, allergy, atopy, asthma, conjunctivitis, periodontitis, rhinitis, otitis media, pharyngitis, tonsillitis, pneumonia, stomach ulcer, gastritis, Crohn's disease, colitis, gout, ankylosing spondylitis, rheumatic fever, lupus, fibromyalgia, psoriatic arthritis, osteoarthritis, rheumatic arthritis, shoulder periarthritis, tendinitis, tenosynovitis, tendonitis, myositis, hepatitis, cystitis, nephritis, Sjogren's syndrome, multiple sclerosis, and acute or chronic inflammatory disorder, but it is not limited thereto.

The pharmaceutical composition of the present invention may further contain a suitable carrier, a vehicle, or a diluent that are commonly used for production of a pharmaceutical composition.

As for the pharmaceutical administration form of CSP-4 peptide of the present invention, CSP-4 peptide can be used in pharmaceutically acceptable salt form thereof. CSP-4 peptide can be also used either singly or as a complex or a suitable group with other pharmaceutically active compounds.

The CSP-4 peptide-containing pharmaceutical composition of the present invention can be used after formulating it, according to a common method, in the form of oral formulation such as a powder preparation, a granule, a tablet, a capsule, a suspension, an emulsion, a syrup, or an aerosol, or a preparation for external use, a suppository, or a sterilized injection solution. Examples of the carrier, vehicle, and diluent which may be included in the pharmaceutical composition containing CSP-4 peptide include various compounds and mixture including lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methy hydroxybenzoate, propyl hydroxybenzoate, talc, magnesium stearate, and mineral oil. In case of having a preparation, production is made by using a diluent or a vehicle such as filler, bulking agent, binding agent, moisturizing agent, disintegrating agent, or surfactant that are commonly used for producing a preparation. As for the solid preparation for oral administration, a tablet, a pill, a powder preparation, a granule, a capsule or the like are included, and such solid preparation is produced by mixing CSP-4 peptide with at least one vehicle such as starch, calcium carbonate, sucrose, lactose, or gelatin. Furthermore, other than simple vehicles, a lubricating agent such as magnesium stearate or talc is also used. As for the liquid preparation for oral administration, a suspension, a solution preparation for internal use, an emulsion, a syrup preparation, or the like can be mentioned. Other than water or liquid paraffin as a commonly used simple diluent, various kinds of a vehicle such as moisturizing agent, sweetening agent, aromatic agent, or preservatives may be included therein. Examples of a preparation for parenteral administration include a sterilized aqueous solution, a non-soluble agent, a suspending agent, an oil agent, a freeze-drying agent, and a suppository agent. As a water insoluble solvent or a suspending agent, propylene glycol, polyethylene glycol, or vegetable oil such as olive oil, and injectable ester such as ethylolate can be used. As a base for a suppository, WITEPSOL, macrogol, TWEEN 61, cacao fat, laurin fat, glycerogelatin, or the like can be used.

Preferable dose of CSP-4 peptide of the present invention may vary depending on symptoms and body weight of a patient, severeness of disease, pharmaceutical form, administration route and period, but it can be suitably selected by a person skilled in the pertinent art. However, to have a preferred effect, CSP-4 peptide of the present invention is administered in an amount of 0.0001 to 100 mg/kg, and preferably 0.001 to 100 mg/kg per day. The administration can be made once a day, or several times a day. The scope of the present invention is not limited in any sense by the aforementioned dose.

CSP-4 peptide of the present invention can be administered to a mammal like rat, mouse, livestock, and human via various routes. As for the type of administration, any kind of administration type is expected, and the administration can be made by oral, rectal, intravenous, muscular, subcutaneous, endometrium injection, or intracerebroventricular injection, for example.

The present invention further provides an anti-inflammatory composition which comprises, as an effective ingredient, a clavaspirin peptide analogue consisting of the amino acid sequence of SEQ ID NO: 2.

Since the clavaspirin peptide analogue of the present has an excellent anti-inflammatory effect without generating any side effect, the peptide may function as a favorable effective ingredient of the aforementioned anti-inflammatory composition.

The present invention further provides a cosmetic composition for preventing or alleviating an inflammatory disorder which comprises, as an effective ingredient, a clavaspirin peptide analogue consisting of the amino acid sequence of SEQ ID NO: 2.

According to one embodiment of the present invention, when CSP-4 peptide is applied on the skin of a mouse with induced inflammation, an excellent anti-inflammatory effect was shown without exhibiting any side effect. Thus, the peptide may function as a favorable effective ingredient of the aforementioned cosmetic composition for alleviating inflammation.

The cosmetic composition of the present invention may be prepared as any formulation such as a skin ointment for external use, cream, a softening cosmetic water, a nutritive cosmetic water, a pack, an essence, a hair tonic agent, a shampoo, a rinse, a hair conditioner, a hair treatment, a skin lotion, a skin softener, a skin toner, an astringent, a lotion, a milk lotion, a moisturizing lotion, a nutritive lotion, a massage cream, a nutritive cream, a moisturizing cream, a hand cream, a foundation, a nutritive essence, a soap, a cleansing foam, a cleansing lotion, a cleansing cream, a body lotion, or a body cleanser. The composition for each of those formulations may have various bases and additives that are necessary and suitable for production of the formulation, and the type and amount of those components can be easily determined by a person skilled in the pertinent art.

When the formulation of the present invention is paste, cream, or gel, animal oil, vegetable oil, wax, paraffin, starch, tragacanth, cellulose derivatives, polyethylene glycol, silicone, bentonite, silica, talc, or zinc oxide can be used as a carrier component.

When the formulation of the present invention is powder or spray, lactose, talc, silica, aluminum hydroxide, calcium silicate, or polyamide powder can be used as a carrier component. When the formulation is spray, in particular, a propellant such as chlorofluorohydrocarbon, propane/butane, or dimethyl ether may be additionally included.

When the formulation of the present invention is a solution or an emulsion, a solvent, a solubilizing agent, or an emulsifying agent is used as a carrier component, and examples thereof include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, and fatty acid ester of sorbitan.

When the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, or tragacanth can be used as a carrier component.

When the formulation of the present invention is a surfactant-containing cleanser, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkyl amodi betaine, aliphatic alcohol, fatty acid glyceride, fatty acid diethanolamine, vegetable oil, lanolin derivatives, or ethoxylated glycerol fatty acid ester can be used as a carrier component.

The present invention still further provides an antimicrobial composition for use against one or more selected from a group consisting of Staphylococcus aureus, Bacillus subtilis, and MRSA (methicillin-resistant Staphylococcus aureus) which comprises, as an effective ingredient, a clavaspirin peptide analogue consisting of the amino acid sequence of SEQ ID NO: 2.

The expression “antimicrobial” means an ability of lowering, preventing, inhibiting, or eradicating the growth or survival of microorganisms at certain concentration, and it preferably means “anti-bacterial.” The bacteria are preferably Gram-negative bacteria, Gram-positive bacteria, or antibiotics-resistant bacteria. More preferably, Gram-negative bacteria are Escherichia coli or Proteus vulgaris, Gram-positive bacteria are Listeria monocytogenes, Staphylococcus epidermidis, Bacillus subtilis, or Staphylococcus aureus, and the antibiotics-resistant bacteria are MRSA (methicillin-resistant Staphylococcus aureus), but they are not limited thereto.

Hereinbelow, the present invention is explained in greater detail in view of the Examples. However, it is evident that the following Examples are given only for exemplification of the present invention and by no means the present invention is limited to the following Examples.

### EXAMPLES

### Example 1: Synthesis, Isolation, and Purification of Peptide

According to the solution phase peptide synthesis by Merrifield (Merrifield, R B., J. Am. Chem. Soc., 85:2149-2154, 1963), the inventors of the present invention substituted isoleucines (I) at the 9^{th} and the 12^{th} positions of the hydrophobic part of the peptide, which has the amino acid sequence of SEQ ID NO: 1 and is described as clavaspirin as a mother peptide, with lysine (K) to synthesize CSP-4 peptide (SEQ ID NO: 2) (test group 1) (Table 1).

Specifically, as for the peptide in which the peptide designed in the present invention has a carboxy terminal in NH₂ form, a rink amide MBHA-resin was used as a starting material, and as for the peptide having a carboxy terminal in OH form, a Fmoc (9-fluorenylmethoxycarbonyl)-amino acid-Wang resin was used as a starting material.

Peptide chain extension based on Fmoc-amino acid coupling was carried out by DCC (N-hydroxybenzotrizole (HOBt)-dicyclo-hexycarbodiimide) method. After coupling Fmoc-amino acid at the terminal amino acid of each peptide, the Fmoc group is removed by using NMP (20% piperidine/N-methyl pyrrolidone) solution. Then, after washing several times with NMP and DCM (dichloromethane), drying with nitrogen gas was carried out. Then, a solution in which TFA (trifluoroacetic acid), phenol, thioanisole, H₂O, and triisopropylsilane are mixed at ratio of 85:5:5:2.5:2.5 (v/v) was added thereto followed by reaction for 2 to 3 hours to remove the protective group and separate the peptide from resin. Then, the peptide was allowed to precipitate in diethyl ether. The crude peptide obtained by the above method was purified by using a purification type reverse phase (RP)-HPLC column (Delta Pak, C₁₈ 300 Å, 15, 19.0 mm×30 m, Waters, USA) based on acetonitrile gradient containing 0.05% TFA. The synthesized peptide was hydrolyzed with 6 N HCl at 110° C. Then, the resulting residues were concentrated under reduced pressure and dissolved in 0.02 N HCl. The amino acid composition was measured by using an amino acid analyzer (Hitachi 8500 A). As a result of determining the purity of the peptide which has been prepared by the above method, the peptide was confirmed to have purity of 95% or higher, and as a result of comparing the molecular weight of the peptide obtained by MALDI mass analysis (Hill et. al., Rapid Commun. Mass Spectrometry, 5: 395, 1991) with the molecular weight that is obtained by calculation from the amino acid sequence, the values were found to be identical to each other (Table 1).

TABLE 1
Sequence, molecular weight, and retention time of
the peptides synthesized in the present invention

| | | | | Reten- |
|---|---|---|---|---|
| Peptide | Amino acid | Sequence | Molecular | tion |
| name | sequence | ID NO. | weight | time |
| Clavaspirin | FLRFIGSVIHGIGH | 1 | 2492.01 | 35.2 |
| | LVHHIGVAL-NH₂ | | | |
| | (SEQ ID NO: 1) | | | |
| CSP-4 | FLRFIGSVKHGKGH | 2 | 2522.04 | 20.2 |
| | LVHHIGVAL-NH₂ | | | |
| | (SEQ ID NO: 2) | | | |

### Example 2: Measurement of Antimicrobial Activity

To compare the antimicrobial activity of the peptide produced by the method of Example 1, the inventors of the present invention measured the growth minimum inhibitory concentration (MIC), which is minimum concentration of the peptide showing no dissociation of bacterial cells.

Specifically, E. coli (Escherichia coli, ATCC 25922), and P. aeruginosa (Pseudomonas aeruginosa, ATCC 15692), S. typhimurium (Salmonella typhimurium, KTCC 1926), and P. vulgaris (Proteus vulgaris, KCTC 2433 as Gram-negative bacteria, and S. aureus (Staphylococcus aureus, ATCC 25923), L. monocytogenes (Listeria monocytogenes, ATCC 19115), B. subtilis (Bacillus subtilis, KCTC 1918), and S. epidermidis (Staphylococcus epidermidis, KCTC 3096) as Gram-positive bacteria were used after obtaining them from American Type Culture Collection (ATCC) or Korean Cell Line Bank. Each bacterial cell line was cultured in LB medium (1% bacto tryptone, 0.5% bacto yeast extract, and 1% sodium chloride; Sigma, USA) to a mid-log phase at pH of 5.5 or 7.4. After that, the cells were diluted in 1% bacto peptone medium (Difco, USA) to have cell body concentration of 5×10⁵ cells/100 μl, and then inoculated to a microtiter plate (NUNC, USA). Thereafter, each of CSP-4 peptide synthesized in Example 1 above and the mother peptide was diluted, 1/2 times for each, at pH of 5.5 or 7.4 in a 96-well plate. After adding the cells to the plate, the cells were cultured for 12 hours at 37° C. Then, the absorbance was measured at a wavelength of 620 nm by using a microtiter plate reader (Merck Elisa reader, Germany) to determine the MIC value of each bacterial strain.

As a result, as it is shown in the following Table 2, CSP-4 peptide of the present invention was confirmed to exhibit a high antimicrobial activity for all bacterial strains (Table 2).

TABLE 2
Antimicrobial activity of antimicrobial peptide against Gram-negative bacteria and Gram-positive bacteria

| | Growth minimum inhibitory concentration (μM) | | | | | | | |
|---|---|---|---|---|---|---|---|---|
| | Gram-negative bacteria | | | | Gram-positive bacteria | | | |
| Peptide | Escherichia coli | P. aeruginosa | S. typhimurium | P. vulgaris | S. aureus | L. monocytogenes | B. subtilis | S. epidermidis |
| Clavaspirin | 2 | 32 | 32 | 8 | 16 | 16 | 16 | 4 |
| CSP-4 | 2 | 32 | 64 | 4 | 8 | 4 | 4 | 4 |

### Example 3: Measurement of Hemolytic Activity

To compare the cytotoxicity among the peptides that are produced by the method of Example 1, erythrocyte hemolytic activity of the synthesized peptide was measured.

Specifically, human erythrocyte was diluted in physiological saline (PBS, pH 7.0) to have concentration of 8%, and clavaspirin, CSP-4, and melittin peptide were serially diluted (i.e., dilution to 1/2 concentration from higher concentration for each dilution) followed by reaction for 1 hour at 37° C. After that, the amount of hemoglobin contained in a supernatant collected by centrifuge at 1,000×g was determined by measuring the absorbance at a wavelength of 414 nm. As a control group to be used as a reference for cell disruption level, the supernatant collected by a treatment with 1% Triton X-100 (Sigma, USA) and a reaction for 1 hour at 37° C. was used for absorbance measurement. By setting the erythrocyte hemolytic activity of Triton X-100 at 100%, the hemolytic activity of the above peptides was calculated using the following mathematical equation 1.

Erythrocyte disrupting ability (hemolysis) (%)=(Absorbance A−Absorbance B)/(Absorbance C−Absorbance B)×100 [Mathematical equation 1]

(in the above equation, Absorbance A indicates the absorbance of a reaction solution treated with each peptide, in which the absorbance is measured at a wavelength of 414 nm; Absorbance B indicates the absorbance of a reaction solution treated with PBS, in which the absorbance is measured at a wavelength of 414 nm; and Absorbance C indicates the absorbance of a reaction solution treated with 1% Triton X-100, in which the absorbance is measured at a wavelength of 414 nm).

As a result, it was found that CSP-4 peptide of the present invention shows almost no hemolytic activity when it is present at concentration of 200 μM or less (Table 3). Accordingly, it was confirmed that the antimicrobial peptide of the present invention causes almost no cytotoxicity.

TABLE 3

| | Erythrocyte disrupting ability % | | | | |
|---|---|---|---|---|---|
| | (concentration of each peptide, μM) | | | | |
| Peptide | 400 | 200 | 100 | 50 | 25 |
| Clavaspirin | 100 | 100 | 100 | 100 | 100 |
| CSP-4 | 10 | 1 | 0 | 0 | 0 |

### Example 4: Determination of Cytotoxicity in Normal Cell Line

To determine the cytotoxicity of the peptide produced by the method of Example 1 in normal cell line, toxicity was measured by using human keratinocyte forming cell line (HaCaT cell line, Dr. NE. Fusenig, Heidelberg, Germany).

Specifically, HaCaT cells which have been cultured in DMEM medium containing 10% FBS (Fetal Bovine Serum) were aliquoted in a 96-well plate (3×10³ cells/well). After culturing them for 24 hours, the cells were subjected to a treatment with CSP-4 peptide at different concentrations (25, 50, 100, 200 or 400 μM/well), followed by reaction for 24 hours in a 5% CO₂ incubator. After the culture, a reaction solution containing 5 mg/ml MTT (Thiazolyl Blue Tetrazolium Bromide) dissolved in physiological saline (PBS) was added in an amount of 20 μl to each well and allowed to react for 4 hours. After that, the supernatant was removed, and, by dissolving the formed MTT crystals by adding 200 μl of DMSO, the absorbance at 560 nm was measured to determine the cell survival ability.

As a result, it was found that the peptide (CSP-4) shows almost no cytotoxicity in human keratinocyte forming cell line (HaCaT cell line) as it is shown in Table 4.

TABLE 4

| | Cell survival ability % | | | | |
|---|---|---|---|---|---|
| | (concentration of each peptide, μM) | | | | |
| Peptide | 400 | 200 | 100 | 50 | 25 |
| Clavaspirin | 0 | 1 | 1 | 3 | 15 |
| CSP-4 | 88 | 100 | 100 | 100 | 100 |

### Example 5: Determination of Antimicrobial Activity Against Antibiotics-Resistant Bacteria

To determine whether or not the synthesized peptide has an antimicrobial activity against highly pathogenic bacteria, various MRSA bacteria (methicillin resistant Staphylococcus aureus) which have been isolated from patients of Chonnam National University Hospital were cultured in LB or TSP to a mid-log phase. The peptide solution which has been serially diluted in a 96-well microtiter plate (NUNC, USA) was added in an amount of 100 μl per well. After adding 100 μl of a medium containing the bacterial strain thereto, the plate was allowed to stand for 24 hours at 37° C. and absorbance at a wavelength of 600 nm was measured. The concentration at which the absorbance is the same as that of a positive control group (i.e., medium without any bacterial strain) was set as minimal inhibitory concentration (MIC). Namely, the concentration exhibiting growth inhibition of 95% or higher corresponds to a MIC value.

TABLE 5

| | MIC (μM) | | | | |
|---|---|---|---|---|---|
| Bacterial strains | Clavaspirin | CSP-4 | Melittin | Erythromycin | Gentamycin |
| S. aureus | 16 | 8 | 2 | 1 | 1 |
| S. aureus MRSA 3126 | 16 | 8 | 2 | 128 | 32 |
| S. aureus MRSA 0027 | 16 | 8 | 2 | >256 | 64 |
| S. aureus MRSA 5157 | 8 | 8 | 2 | 16 | 16 |
| S. aureus MRSA 1635 | 16 | 8 | 2 | >256 | >256 |
| S. aureus MRSA 4761 | 32 | 8 | 4 | >256 | 32 |
| S. aureus MRSA 5159 | 32 | 8 | 8 | 16 | 16 |
| S. aureus MRSA 3-359 | 16 | 8 | 4 | >256 | 64 |
| S. aureus MRSA 2-122 | 8 | 8 | 4 | >256 | 128 |
| S. aureus MRSA 1630 | 8 | 8 | 4 | >256 | 64 |
| S. aureus MRSA 1870 | 8 | 8 | 4 | >256 | 16 |
| S. aureus MRSA 3511 | 8 | 8 | 4 | >256 | >256 |
| S. aureus MRSA 5156 | 8 | 8 | 4 | >256 | >256 |
| S. aureus MRSA 3518 | 8 | 8 | 4 | >256 | >256 |

As a result, as it is shown in Table 5, the antimicrobial activity was shown against highly pathogenic bacteria like antibiotics-resistant bacteria, which originate from human.

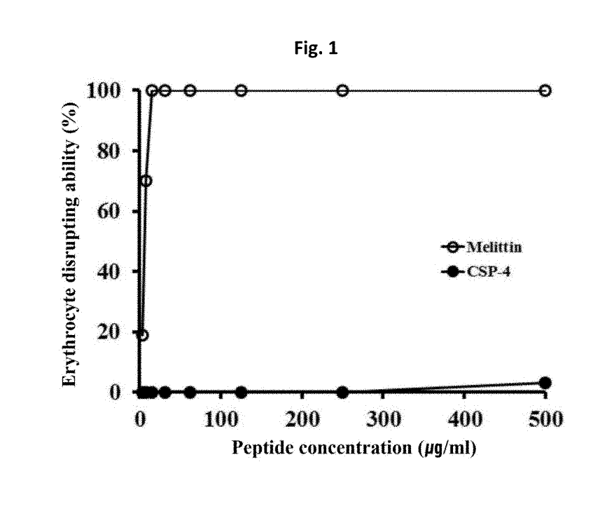

In particular, when comparison is made with melittin, which is known to have an excellent antimicrobial peptide activity, as a comparative group of the test, the activity was found to be the same or lower by 1 or 2 levels. However, due to high cytotoxicity, development of melittin as a pharmaceutical compound is limited. As a result of testing the erythrocyte hemolysis activity as it is shown in FIG. 1, melittin (antimicrobial peptide; ◯) shows hemolysis of almost 100% at 2 μg/ml but the antimicrobial peptide of the present invention (CSP-4; ●) shows hemolysis of less than 1% even at 500 μg/ml. This result indicates that the peptide of the present invention has almost no cytotoxicity (FIG. 1).

Furthermore, by determining that the antibiotics activity is not exhibited at all by erythromycin and gentamycin that are administered to existing patients, it was confirmed that Staphylococcus aureus having antibiotics resistance, which have been isolated from the patients, have no activity with existing antibiotics (Table 5).

### Example 6: Cell Culture and Protein Extraction

Before carrying out cytokine expression test in phagocytes (Raw 264.7 cell) using S. aureus MRSA 0027 as S. aureus having tolerance to antibiotics, phagocytes were cultured at 37° C. in a 5% CO₂ humidified incubator by using DMEM (Dulbecco's modified Eagle medium) supplement added with 100 U/μM penicillin and 10% FCS (fetal calf serum). To carry out a test to determine suppressed expression of TNF-α (Tumor Necrosis factor-α) and IL-1β (Interleukin-1β) as a proinflammatory cytokine, phagocytes were inoculated to a 12-well plate at 1×10⁶ cells/ml and cultured for 1 day in a 5% CO₂ humidified incubator. After that, S. aureus MRSA 0027 which have been cultured in advance were inoculated to the plate (well) at 1×10⁶ cells/ml except the negative control. The remaining plate (well) except the positive control was treated with PELPYK peptide at different concentrations (25, 50, 100 and 200 μg/ml) followed by reaction for 12 hours in a 5% CO₂ humidified incubator (37° C.). Twelve hours later, the supernatant was removed from the 12-well plate, and the adhered phagocytes were collected by using trypsin. In order to obtain pure phagocytes only, the cells were precipitated for 10 minutes at 4,000 rpm, and after removing the supernatant, proteins were extracted by using a protein extracting reagent (PRO-PREP™ protein extraction solution, iNtRON biotechnology).

### Example 7: Determination of Suppressed Expression of TNF-α and IL-1β

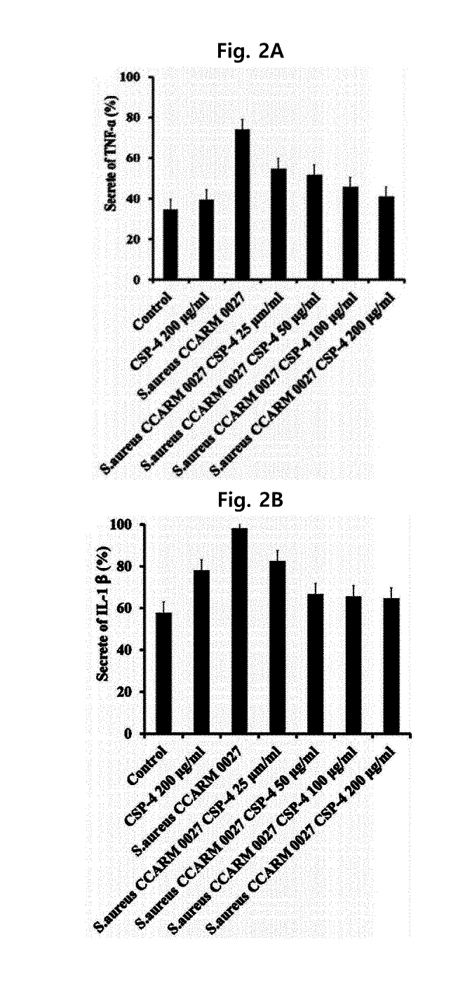

Each protein extracted by using a protein extracting reagent was quantified based on Bradford method, which is a protein quantification method. After having a reaction by using TNF-α and IL-1β as a proinflammatory cytokine (KOMABIOTECH, Korea), ELISA measurement was carried out using absorbance at 450 nm. Cultured phagocytes (RAW 264.7), which have been counted (5×10⁵ cells/ml), cultured for 1 hour in a 5% CO₂ incubator, and inoculated with 1×10⁶ cells/ml of S. aureus MRSA 0027 known to cause dermatitis, were treated with the antimicrobial peptide isolated from a marine organism at concentration of 25, 50, 100, or 200 μg/ml. Then, as a result of determining TNF-α and IL-1β that are generally known as a proinflammatory cytokine, it was found that cytokine is hardly expressed in the cells inoculated only with the antimicrobial peptide when compared to the control group. On the other hand, the cells treated only with the bacteria were induced to have over-expression. However, as a result of carrying out a treatment with CSP-4 peptide at different concentrations for those previously treated with the bacteria, it was confirmed that the expression of TNF-α and IL-1β as a proinflammatory cytokine has decreased in accordance with an increase in the peptide concentration (FIGS. 2A and 2B).

### Example 8: H&E (Haematoxyline and Eosin) Tissue Staining and Immunohistochemical Staining

Skin of a 6-week old hairless mouse was treated with 100 μl of S. aureus MRSA 0027 (1×10⁸ cells/ml) to induce inflammation. Thirty minutes later, the skin was further treated with CSP-4 peptide at 100 μg/ml or 200 μg/ml. From each of a mouse having induced inflammation further treated with the peptide, a mouse treated only with the peptide, and a mouse having induced inflammation, tissues were collected and washed using physiological saline (PBS). Then, the tissues were fixed for 24 hours at room temperature by using 4% PFA (paraformaldehyde). All fixed tissues were dehydrated by increasing the ethanol concentration (i.e., reaction for 2 hours for each), and, after 3 replacements with xylene, each was allowed to react for 1 hour followed by preparation of a paraffin block. The prepared paraffin block sample was used to give a 4 μm section by using microtome (Thermo-scientific, USA), and moisture was completely removed at room temperature. According to H&E staining and immunohistochemical method, a change in tissues and onset of an anti-inflammatory cytokine or TLR2 receptor was determined. Specifically, each tissue prepared as a section was treated with xylene to remove paraffin. As a next step, to remove completely any moisture from the tissue, hydrolysis was carried out (reaction time of 2 hours) while increasing the ethanol concentration. The hydrolyzed tissues were stained by using H&E. In addition, empty spaces between tissues were filled by blocking with 5% BSA (bovine serum albumin), and by using an antibody exhibiting an anti-inflammation response (i.e., anti-TLR-2 mouse (ABfrontier, AB24192), anti-TNF-α, mono, mouse (ABfrontier, AB1793), anti-IL-1β, poly, mouse (ABfrontier, AB1413)), the reaction was allowed to occur for 20 minutes at room temperature such that the antibody can bind to the tissues. Remaining liquids were removed by TBST buffer such that the tissues are not detached, and a secondary antibody linked with green fluorescence (Got anti-mouse IgG (HRP) LF-SA5001-conjugated) was allowed to bind thereto. Next, each tissue was determined by using a fluorescent microscope (I×71, Olympus, Japan).

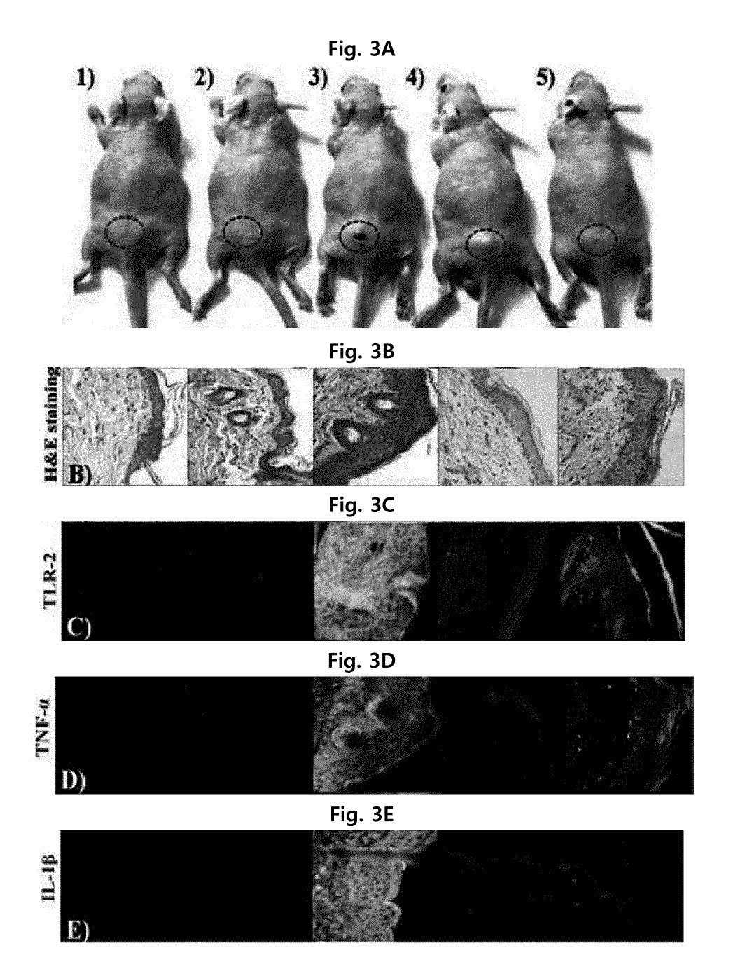

As a result of examining a change in skin surface by the above H&E staining method, it was confirmed that the tissues treated with the bacteria become to have higher thickness and a phenomenon of immune cell aggregation (FIG. 3B). On the contrary, the tissues treated with the peptide exhibited a decreasing inflammation response as the peptide concentration increases. In order to determine more specifically those results, TNF-α and IL-1β, which are a proinflammatory protein, and TLR-2 antibody, which is a receptor related with inflammation exhibition were employed. When the collected tissues were bound with a primary antibody followed by binding with a fluorescence-linked secondary antibody and then observed under a fluorescence microscope, it was found that all proteins are over-expressed when the treatment is carried out only with the bacteria. However, for a case in which the peptide treatment is carried out at different concentrations, it was confirmed that expression of the inflammation-related proteins is hardly induced as the peptide concentration increases (FIGS. 3C, 3D, and 3E). Based on these results, it was recognized that, when dermatitis is induced as a result of infection with microbes, a high anti-inflammatory effect can be obtained by the activity of an antimicrobial peptide (FIGS. 3A to 3E).

### Example 9. Scanning Electron Microscopy (SEM)

From the mouse tissues which have been treated according to the method of above Example 8, a paraffin block was prepared. Then, each sample was used to give a 4 μm section by using microtome (Thermo-scientific, USA), and moisture was completely removed at room temperature. Paraffin was completely removed by using xylene, and the resulting tissues were then coated by using gold powder. Next, a change in the tissues was determined by using scanning electron microscopy (SEM).

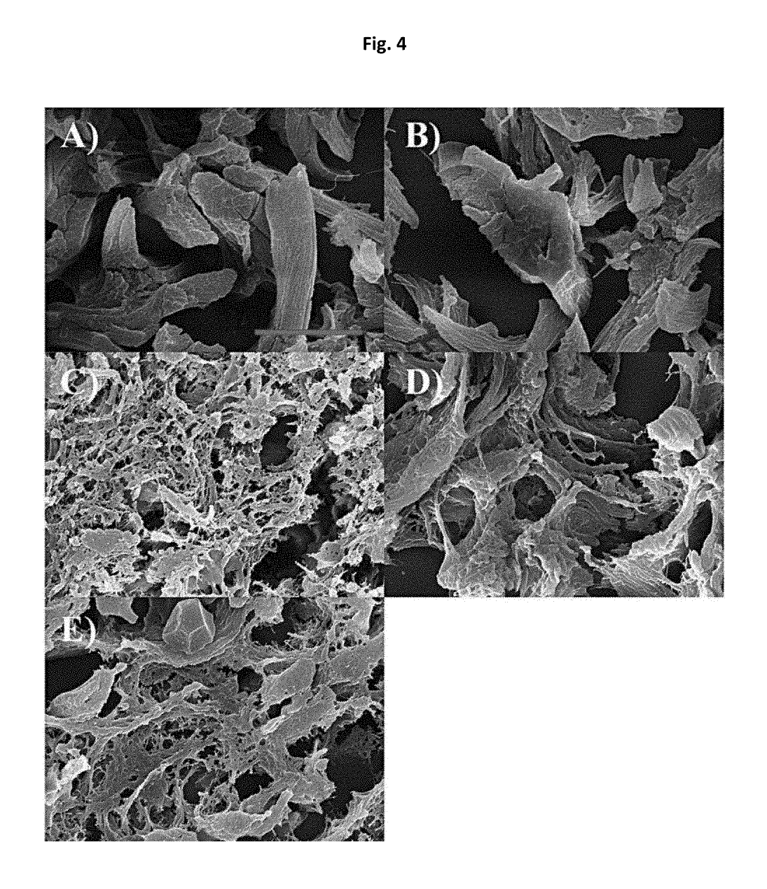

As a result of examining a change in tissues of a hairless mouse which has been induced to have dermatitis (FIG. 4), it was found that no change in mouse skin is induced when the skin is treated only with the antimicrobial peptide of high concentration (200 μg/ml). On the contrary, when the treatment is carried out only with S. aureus CCARM 0027, collagen as mouse skin tissue was significantly disrupted, showing toxicity. When a mouse having dermatitis induced by using S. aureus CCARM 0027 is treated with the antimicrobial peptide of 100 μg/ml or 200 μg/ml, it was found that the collagen disruption in mouse skin is more significantly reduced as the protein concentration is higher. As a result, it was found that the antimicrobial peptide originating from a marine organism exhibits a potent antimicrobial activity against microbes which infect tissues, and thus the antimicrobial peptide can be used for prevention of an occurrence of dermatitis (FIG. 4).

### Example 10. Western Blot Analysis

Skin of a 6-week old hairless mouse was treated with 100 μl of S. aureus MRSA 0027 (1×10⁸ cells/ml) to induce inflammation followed by a treatment with the antimicrobial peptide at different concentrations. Then, after collecting tissues from the mouse, protein extraction was carried out by using a protein extraction reagent. Concentration of the extracted proteins was quantified by Bradford method, and the quantified sample was electrophoresed on a 15% acrylamide gel by SDS-PAGE for 3 hours. Then, the electrophoresed proteins were transferred onto a PVDF membrane (Bio-Rad, USA) for 1 hour at 90 V, 4° C. After that, each primary antibody (anti-GAPDH (Santa Cruz Biotechnology, LF-PA0018), anti-TLR2, mono, mouse (ABfrontier, AB24192), anti-NF-κB, poly, mouse (Santa Cruz Biotechnology, SC-71675), anti-TNF-α, mono, mouse (ABfrontier, AB1793), anti-IL-1β, poly, mouse (ABfrontier, AB1413) and anti-Cathelicidin, poly, mouse (ABfrontier, AB93357)) was added to 5% skim milk blocking solution, and binding was allowed to occur for 1 day at 4° C. The membrane bound with a primary antibody was washed with TBST buffer. Next, by having a reaction for 2 hours at room temperature with a secondary antibody (Goat anti-rabbit IgG (HRP) LF-SA5002 and Goat anti-mouse IgG (HRP) LF-SA5001-conjugated), binding of a secondary antibody to the membrane bound with a primary antibody was allowed to occur. The membrane further bound with a secondary antibody was washed with TBST buffer to remove completely any non-bound materials, and according to the HRP chromogenic reaction linked to a secondary antibody (western blot detection kit, ABfrontier, LF-QC0103), expression level of the inflammatory proteins was determined.

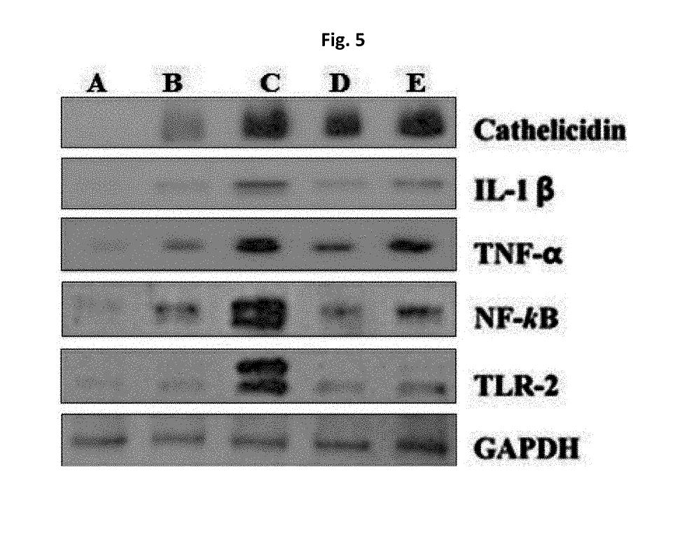

As a result of determining the proteins that are related with an inflammatory response based on the above Western blot analysis, it was found that, in the tissues having induced skin inflammation, over-expression of signal transduction proteins (TLR-2, NF-kB), cytokines (TNF-α, IL-1β), and cathelicidin antimicrobial peptide is shown compared to a control group, but those inflammation-related proteins are reduced according to an increase in treatment concentration of the antimicrobial peptide of the present invention (FIG. 5). Accordingly, it was confirmed that the antimicrobial peptide which has been modified after being isolated from a marine organism has a potent activity of suppressing inflammation.

### REFERENCE TO SEQUENCE LISTING

Pursuant to 37 C.F.R. § 1.821(c) or (e), an ASCII text file containing an electronic version of the Sequence Listing has been submitted concomitant with this application via EFS-Web, the contents of which are hereby incorporated by reference.

1. A composition for preventing or alleviating an inflammatory disorder, comprising a clavaspirin peptide analogue having the amino acid sequence of SEQ ID NO: 2 as an effective ingredient.

2. A functional health food comprising the composition of claim 1.

3-5. (canceled)

6. A pharmaceutical comprising the composition of claim 1.

7-10. (canceled)

11. An anti-inflammatory drug comprising the composition of claim 1.

12-13. (canceled)

14. A method of preventing or alleviating an inflammatory disorder, comprising administering or applying to a subject in need thereof a composition comprising a clavaspirin peptide analogue having the amino acid sequence of SEQ ID NO: 2 as an effective ingredient.

15. The method of claim 14, wherein the inflammatory disorder is caused by bacteria.

16. The method of claim 15, wherein the bacteria are at least one selected from the group consisting of Gram-negative bacteria, Gram-positive bacteria and antibiotics-resistant bacteria.

17. The method of claim 16, wherein Gram-negative bacteria are Escherichia coli or Proteus vulgaris;
Gram-positive bacteria are Listeria monocytogenes, Staphylococcus epidermidis, Bacillus subtilis, or Staphylococcus aureus; and
the antibiotics-resistant bacteria are MRSA (methicillin-resistant Staphylococcus aureus).

18. The method of claim 14, wherein the composition inhibits expression of TNF-α, IL-1β, NF-κB, or TLR-2.

19. The method of claim 14, wherein the composition is included in a functional health food.

20. The method of claim 14, wherein the composition is included in a pharmaceutical.

21. The method of claim 14, wherein the composition is included in an anti-inflammatory drug.

22. The method of claim 14, wherein the composition is included in an antimicrobial.

23. The method of claim 14, wherein the composition is included in a cosmetic.

24. The method of claim 14, wherein the inflammatory disorder is at least one selected from a group consisting of dermatitis, allergy, atopy, asthma, conjunctivitis, periodontitis, rhinitis, otitis media, pharyngitis, tonsillitis, pneumonia, stomach ulcer, gastritis, Crohn's disease, colitis, gout, ankylosing spondylitis, rheumatic fever, lupus, fibromyalgia, psoriatic arthritis, osteoarthritis, rheumatic arthritis, shoulder periarthritis, tendinitis, tenosynovitis, tendonitis, myositis, hepatitis, cystitis, nephritis, Sjogren's syndrome, multiple sclerosis, and acute or chronic inflammatory disorder.

25. A method of lowering, preventing, inhibiting, or eradicating a growth or survival of a microorganism, the method comprising using against a microorganism a composition comprising a clavaspirin peptide analogue having the amino acid sequence of SEQ ID NO: 2 as an effective ingredient.

26. The method of claim 25, wherein the microorganism is selected from the group consisting of Staphylococcus aureus, Bacillus subtilis, and MRSA (methicillin-resistant Staphylococcus aureus).
